(19) United States
(12) Patent Application Publication
(10) Pub. No.: US 2026/0060192 A1
(43) Pub. Date: Mar. 5, 2026

(54) METHOD FOR IMPROVING FLOWERING OF A PLANT OF THE FAMILY RUBIACEAE

(71) Applicant: SOCIÉTÉ DES PRODUITS NESTLÉ S.A., Vevey (CH)

(72) Inventors: David BRETON, La Membrolle sur Choisille (FR); Mathieu SIMON, La Ville aux Dame (FR); Rafael CHAN, Tours (FR)

(21) Appl. No.: 18/997,732

(22) PCT Filed: Jul. 25, 2023
(86) PCT No.: PCT/EP2023/070639

§ 371 Date: Jan. 22, 2025

(30) Foreign Application Priority Data
Jul. 26, 2022 (EP) 22187025.6

(51) Int. Cl. A01H 3/00; A01H 3/02; A01H 5/02; A01H 6/76

(52) U.S. Cl. CPC A01H 3/00; A01H 3/02; A01H 5/02; A01H 6/76

(57) ABSTRACT

The invention relates to a method for accelerating the time to flowering of a plant and/or for increasing the amount of flowers and/or fruits produced by said plant of the family rubiaceae, the method comprising providing a plant seed or plant part which is capable of regeneration, cultivating the same to a stage in which a cultivated shoot is obtained that comprises a fork from which an orthotropic shoot and at least two plagiotropic branches have developed, pruning the cultivated shoot at said fork by removing at least the orthotropic shoot and maintaining at least one plagiotropic branch to obtain a pruned shoot, cultivating the pruned shoot until flowers form on the at least one plagiotropic branch to obtain a flowered shoot.

### CROSS REFERENCE TO RELATED APPLICATIONS

The present application is a National Stage of International Application No. PCT/EP2023/070639 filed Jul. 25, 2023, which claims priority to European Patent Application No. 22187025.6 filed Jul. 26, 2022, the entire contents of which are incorporated herein by reference.

### TECHNICAL FIELD OF THE INVENTION

The invention relates to a method for improving the flowering, in particular accelerating the time to flowering of and/or for increasing the number of flowers and/or fruits produced by a plant of the family rubiaceae, and to flowering plants obtainable by the method.

### BACKGROUND OF THE INVENTION

Plants of the genus Coffea, also called coffee plants, rank as one of the world's most valuable and widely traded commodity crops. Under natural conditions, it takes 2 to 3 years after seed germination for a coffee plant to first produce flowers, with subsequent flower production only approximately once a year. For a breeder, it is necessary to pass this vegetative growth phase to be able to perform controlled crosses and obtain new genetic germplasm. Furthermore, agronomic evaluations are usually based on data collected on 3 subsequent crops of the same coffee plant. In total, this results in a single breeding cycle taking approx. 5 to 6 years to complete. In some instances, e.g., in the case of inbred parental lines, several breeding cycles have to be completed before offspring with satisfying attributes is obtained.

Producing coffee seeds using controlled crosses requires planting parental trees in a seed garden and cultivating the plants for two to three years until flowers form. In manual labor, pollen from a parent plant is collected and applied to the pistil of a different plant which serves as a mother plant.

Hybrid seeds produced by seed gardens require geographical or physical isolation from other coffee plantations to ensure the genetic purity of the obtained seeds. Once planted, seed garden trees stay for years in the same plot that is able to deliver a limited amount of coffee hybrid varieties. Moreover, synchronization of the flowering events is key between parents to be able to obtain fruits and seeds. In some cases, this is not occurring under natural conditions, thus preventing crossing.

In light of the long breeding cycle of coffee plants, and of other plants of the family of rubiaceae in general, methods for accelerating the flowering have been proposed. These involve treatment of the plants with hormones and/or introducing transgenes:

EP 0 190 885 A2 describes treatment of woody angiosperms with rapidly metabolizable gibberellin to promote early flowering, optionally together with natural cytokinin zeatin, to reduce the severity of biennial bearing.

WO 2004/010767 A2 describes over-expressing PtM3 or PtM4 in the reproductive tissues of plants to accelerate flowering.

WO 2014/007400 A1 describes introducing transgenic geranyl pyrophosphate synthase large subunit protein (derived from goldfish) to produce plants that flower at an early stage.

However, these methods are limited by several drawbacks. Hormone treatment of plants can have unforeseeable side effects, thus hampering the reliability of the breeding scheme. Genetically modified plants face strict regulations and may be avoided by consumers and farmers. Moreover, breeding genetically modified plants requires special precautions in order to comply with official conditions of containment and many methods of introducing transgenes into plants are laborious.

### SUMMARY OF THE INVENTION

Against this background, the object underlying the invention was to provide a reliable and easy-to-perform method for accelerating the flowering in plants and/or for increasing the number of flowers and/or fruits produced by said plants. Preferably, the method should not involve the use of hormones or transgenes.

The invention achieves this object by the features recited in the claims and further herein. In particular, the invention provides a method for accelerating the time to flowering of a plant and/or for increasing the number of flowers and/or fruits produced by said plant, which preferably is the first flowering of the plant, and which plant is of the family rubiaceae. The method comprises the steps of
a. providing a plant seed or plant part which is capable of regeneration,
b. cultivating the same to a stage in which a cultivated shoot is obtained that comprises a fork from which an orthotropic shoot and at least one plagiotropic branch have developed,
c. pruning the cultivated shoot at said fork by removing at least the orthotropic shoot and maintaining at least one plagiotropic branch to obtain a pruned shoot,
d. cultivating the pruned shoot until flowers form on the at least one plagiotropic branch to obtain a flowered shoot.

The invention also provides advantageous uses, in particular the use of the flowered plants obtained in an accelerated manner according to the invention for producing fruit and/or seeds or in a breeding program, and the use of pruning for accelerating the time of flowering of a plant of the family rubiaceae and/or the use of pruning for increasing the number of flowers and/or fruits produced by said plant. As in the method of the invention, the pruning is carried out on a cultivated shoot of the plant at a growth stage in which it has branched into an orthotropic shoot and at least one plagiotropic branch, and comprises removing at least the orthotropic shoot and maintaining at least one plagiotropic branch.

The method allows for a reliable induction of early flowering of plants of the family rubiaceae without genetic manipulation and does not require hormone treatment of the plant.

### DETAILED DESCRIPTION OF THE INVENTION

The method of the invention applies to plants of the family rubiaceae in general. Rubiaceae are a family of flowering plants, commonly known as the coffee, madder, or bedstraw family. Economically important genera include Coffea, the source of coffee, Cinchona, the source of the antimalarial alkaloid quinine, some dye plants (e.g., Rubia), and ornamental cultivars (e.g., Gardenia, Ixora, Pentas). Preferably, the plant of the family rubiaceae is a terrestrial tree, in particular one characterized by opposite leaves with interpetiolar stipules and sympetalous actinomorphic flowers. More preferably the plant is of the genus Coffea, including but not limited to the species Coffea arabica, Coffea benghalensis, Coffea canephora, Coffea liberica, Coffea racemosa, and any species derived from the aforementioned.

In a preferred embodiment, the plant is a variant of Coffea arabica, in particular one exhibiting male sterility. In this embodiment, the plant and method offer the advantage that hybrid offspring can be generated more easily, in greater quantity and faster, e.g., by crossing a flower of a pruned shoot according to the invention with pollen from a male donor plant, thus avoiding undesired self-pollination or unwanted cross-pollination. Hybrid plants generated from pure lines of Coffea arabica are on average more vigorous and sturdier than plants generated by self-pollination. The invention allows to increase the number of flowers obtained, to increase the quantity of beans, and to generate more vigorous and sturdier plants. In one embodiment, the plant can be an allotetraploid variant.

The development of plants of the family rubiaceae encompasses the longitudinal growth of its stem. When a certain length of the stem is reached, a fork is formed where the stem branches out into an orthotropic shoot and at least one plagiotropic branch. Upon further development, in particular when the original orthotropic shoot of the plant has been pruned, the plagiotropic branches can form further forks which may branch out into additional branches and/or shoots. The term “fork” as used herein means the structure at the branching point of the stem that forms during outgrowth of the at least one plagiotropic branch.

Along the plagiotropic branches, buds may form which are undeveloped shoots. Buds can be classified as vegetative buds, i.e. buds that develop into leaves, or floral buds, i.e. buds that develop into flowers. Buds are formed at sprouting structures of the plagiotropic branch, which are called “nodes”.

The method of the invention and plant obtained thereby can be used directly for producing fruit. However, particular advantages can be achieved when the plant is used for accelerated production of seeds and/or pollen in a breeding program. Accordingly, when the plant is used in a breeding program, subsequently to completing the method, the produced pollen and/or pistil and/or seeds can be used to propagate the plant, e.g., in a conventional manner involving manual crossing. In this regard, it is an advantage of the method of the invention that the number of floral buds per plagiotropic branch and, more importantly, the number of pollinatable flowers per plagiotropic branch, preferably per plant, can be significantly increased.

When, instead or in addition, the plant fruit is desired for agronomical purposes or for phenotypic assessment in the realm of a breeding program, the plant can be cultivated further until fruits develop, and the fruits can be collected and further studied or processed after completing the method steps of the invention. In this regard, it is a particular advantage of the method of the invention that the number of fruits per plant that can be obtained after a short period of cultivating can be significantly and reliably increased.

In the context of the present method, the term ‘plant’ can refer to a plant seed or plant seedling or plant part which is capable of regeneration, i.e. able to grow. The term “plant part” can in particular refer to a rooted cutting or a cultivated shoot.

In general, the method according to the invention is initiated by step a., wherein a plant seed, seedling or plant part which is capable of regeneration is provided. Preferably, the plant seed or plant part is provided in soil or hydroculture or other conditions that allow for the cultivation of the plant seed or plant part in step b, e.g., soil-less conditions.

“Cultivating” as used herein means any conditions that allow the plant seed to germinate and for plant growth into a cultivated shoot to occur or that allow the plant part to grow further so as to produce a cultivated shoot. These conditions are well-known to those skilled in the art of horticulture of rubiaceae plants, in particular coffee. The plant seed or plant part is generally cultivated in horticulture, preferably not in a field or garden, but under more controlled conditions, such as roofed and/or indoors and/or in a greenhouse facility.

The general aim of the method of the invention is to shorten the time to flowering. While the pruning in step c. is critical specifically for inducing early flowering, the aim of accelerating the process as a whole is assisted very much if the cultivating in step b. and/or d. of the method of the invention is performed under optimal conditions. In particular, such conditions of horticulture should be chosen that allow for fast outgrowth of the shoot and branches by optimized temperature, humidity, light, irrigation, CO₂ concentration, and fertilization.

The cultivating in step b. and/or d. of the method of the invention generally comprises growing at conditions which are beneficial for the plant. The cultivating can comprise growing at a temperature of 16 to 35° C., preferably at least 18° C. or at least 20° C. Preferably, the temperature is up to 32° C., up to 30° C., up to 28° C., up to 26° C., up to 25° C. or up to 22° C. on average. In particular, the research leading up to the invention has shown that the optimum temperature can depend on the plant species. E.g. for plants of the species Coffea arabica, the temperature can be 16-28° C.; for Coffea robusta plants, the temperature can be 24-35° C.

The cultivating can further comprise growing at 60 to 95% relative humidity (RH), preferably at least 65% RH or at least 70% RH, and up to 90% RH, up to 85% RH, up to 80% RH or up to 75% RH, e.g., 70 to 80% RH, e.g., 72% RH.

The cultivating in step b. and/or step d. of the method of the invention usually comprises irrigation and preferably comprises fertigation, i.e. providing irrigation by a fertigation medium which comprises or consists of water supplemented with fertilizer. The fertilizer is preferably water-soluble. The irrigation or fertigation can particularly advantageously be carried out by means of sprinklers, by drip irrigation, or, e.g., in a hydroponic system. Water or the fertigation medium can be applied to the plant continuously, or, e.g., in intervals of once per day, preferably twice per day, or, e.g., approximately every 8 hours, every 6 hours, every 4 hours, every 2 hours, every 1 hour or every 30 minutes.

Preferably, the fertigation medium comprises fertilizer that contains all three primary nutrients (i.e. N-, P- and K-containing compounds). The skilled person readily understands that the fertilizer can further contain further nutrients such as, e.g., Mg, S, Ca, as well as micronutrients, e.g., Cu, Mn, Ni, Zn, Fe. The fertilizer can, e.g., be in powdery or liquid form. Preferably, the fertilizer comprises 5 to 60 wt-% N, 5 to 60 wt.-% P and 5 to 60 wt.-% K, wherein the percentage is understood as the weight of elemental N, P and K as a fraction of the total weight of the fertilizer. Good results were achieved with a powdery fertilizer in step b. comprising 20 wt.-% N, 20 wt.-% P, 20 wt-% K. During step d. of the method of the invention, the fertigation medium can, e.g., comprise fertilizer containing more K, e.g., 8-15% N, 16-25% P, 28-40% K. It has been found that fertilizer containing an increased amount of K can improve fruit formation in the plants. Generally, the fertilizer can be contained in the fertigation medium at a concentration of 0.1 to 10 g/L. The fertigation medium can have a conductivity of 0.1 to 5 mS, preferably 0.2 to 2 mS, more preferably 0.5 to 1 mS, e.g., 0.8 mS.

Preferably, the cultivating in step b. and/or d. of the method of the invention takes place in form of vertical gardening and in facilities that allow for vertical gardening. “Vertical gardening” as used herein means horticulture in vertically stacked layers of culturing vessels, e.g., flowerpots and/or under soil-less growing conditions, e.g., using hydroponic techniques.

Optionally, during the cultivating in step b. and/or step d. of the method, the lighting is controlled. The light applied to the plant can comprise natural light and/or artificial light.

Artificial light useful in the method of the invention is generally generated by a light source emitting light in the spectral range of 400 to 750 nm, e.g., by a sodium-vapor lamp, preferably by an LED grow lamp emitting light at wavelengths corresponding to the absorption maxima of plant chlorophyll, especially one that emits a spectrum rich in blue, red, and far-red wavelengths.

Preferably, the lighting during the cultivating in step b. and/or step d. is set up such that the plant receives natural and/or artificial light having a photosynthetic photon flux density of 80 to 1500 μmol m⁻² s⁻¹, e.g., by positioning the light source at an appropriate distance from the plant, e.g., at maximum 50 cm, preferably at maximum 30 cm or at maximum 20 cm, e.g., 2 to 50 cm or 2 to 40 cm. In step d., the distance is preferably measured between the light source and the at least one plagiotropic branch. It is especially preferred that the cultivating in step d. comprises stimulating specifically the remaining at least one plagiotropic branch by exposure to a light source shining on said at least one plagiotropic branch. In contrast, conventional horticulture of coffee plants usually involves light sources placed at a distance of 1-3 m from the plagiotropic branches, e.g., due to conventional overhead light sources and/or the orthotropic shoot or additional branches along the orthotropic shoot blocking access to the plagiotropic branch. This is because the research underlying the invention showed that especially optimized lighting directed to the plagiotropic branch during cultivating in step d. can further accelerate the time to flowering and/or increase the number of flowers and/or fruits produced by the plant.

In a preferred embodiment, the lighting during cultivating is controlled in that the photoperiod, i.e. the amount of time per day that the plant is exposed to light during the cultivating of step b. and/or step d., is controlled. It has been found that the photoperiod seems to play a crucial role in timing the yearly flowering under outside natural growing conditions and that controlling the length of the photoperiod during the cultivating steps b. and d. of the method of the invention can further accelerate the time to flowering of plants of the rubiaceae family.

Good results were achieved when the length of the photoperiod was controlled for each of step b. and step d., independently from one another or identically, to 8-14 h per day, e.g., 8-10 h per day, 10-12 h per day, 12-14 h per day, 8-12 h per day, or 10-14 h per day. In one embodiment, the length of the photoperiod during the cultivating of step b. is longer than the photoperiod during the cultivating of step d., e.g., 10-14 h in step b. and 8-12 h in step d. of the method of the invention.

In a further embodiment, the cultivating of step d. can comprise altering the length of the photoperiod. Therein, in a first phase the photoperiod can have a first length and in a second phase a second length, which differs from the first length, e.g., by at least 2 hours. E.g., during the first phase the photoperiod can be 8-9 h per day and during the second phase the photoperiod can be 11-12 h per day. Said first phase and second phase, independently from one another or identically, can each have a duration of 1 to 5 months, in particular 2 to 4 months, e.g., 3 months. Therein, the length of the photoperiod can be repeatedly, e.g., periodically, altered between the first phase and the second phase, e.g., every 3 months. It has been found that by altering the photoperiod during a second phase of the cultivating of step d., even earlier flowering of the plant can be induced.

In one embodiment, the light applied to the plant in step b. and/or step d. is only artificial light, such that step b. and/or step d. is carried out while wholly or partially blocking off natural light at all times or only at times outside the photoperiod, i.e. only while the artificial light source is turned off, e.g., by encasing the plant in a light-tight housing or foil or by performing the method of the invention under indoor conditions that effectively prevent exposure of the plant to natural light. This can, e.g., be achieved by horticulture in a container or room with artificial lightning but without windows.

In an alternative embodiment, the light applied to the plant in step b. and/or step d. comprises or is only natural light. Also in this embodiment, lighting can be controlled by blocking off natural light at times outside the photoperiod, e.g., by encasing the plant in a light-tight housing or foil.

By controlling the lighting, the method has the particular advantage that flower induction in the plants, i.e. the exact timepoint when flowers form, can be controlled more precisely, especially far more precisely than in conventional methods.

A further advantage of the method of the invention is that it allows for inducing flowering outside of the natural flowering time in the field, which in the case of coffee plants is linked to the rain season and/or to the natural photoperiod. An advantage of being able to control flower induction by way of the method of the invention is that flower induction can be synchronized between different plants. This is particularly advantageous in breeding application as it allows for easier propagation or crossing of the plants.

In general, the cultivating in step b. is carried out until a stage is reached in which a cultivated shoot is obtained that comprises a fork from which an orthotropic shoot and at least two plagiotropic branches have developed. This growth stage is well discernable for plants of the family rubiaceae as it corresponds to the natural initial branching event. E.g. when grown from seeds, plants of the family rubiaceae first grow into an orthotropic shoot, which then forms a fork from which one or two plagiotropic branches grow outward. In the natural setting, the orthotropic shoot then continues its growth and subsequently further plagiotropic branches grow out of new forks formed further upward on the orthotropic shoot. In this way, the plant eventually becomes a full-grown tree.

In general, in particular from experience with the genera Coffea, the cultivating in step b., i.e. the time that is required from the seed or shoot stage to the formation of the first two plagiotropic branches, is 2-6 or 3-4 months of cultivating in step b. But the duration of the cultivating in step b. of course depends on the type and growth stage of the starting material provided in step a. and the cultivating conditions chosen in step b.

The cultivated shoot that is obtained in step b. and pruned in step c. generally comprises a fork, preferably exactly one fork, from which an orthotropic shoot and at least one plagiotropic branch have developed. In a preferred embodiment, the fork used for pruning in step c. is the first fork that has developed in the cultivated shoot during the cultivating in step b. During the development of rubiaceae plants, especially of Coffea plants, the first fork develops one or two plagiotropic branches and one orthotropic shoot.

The pruning according to step c. of the method comprises cutting of the orthotropic shoot and all but at least one plagiotropic branch, preferably all but exactly one plagiotropic branch. The position of the cut depends on the size of the plant. For coffee plants, good results were achieved when the cut was made at a distance of at maximum 5 cm from the fork, preferably at maximum 2 cm, more preferably at maximum 1 cm or at maximum 0.5 or 0.2 cm from the fork. The cut should not be so close to the fork that the fork itself is harmed. This can usually be achieved by distancing the cut by at least 0.2 cm from the fork.

Generally, the pruning according to step c. of the method is carried out at maximum one month after the fork has formed, preferably at maximum 3 weeks, at maximum 2 weeks or at maximum 1 week, even more preferably less than one week after the fork has formed.

Optionally, the method in step d. further comprises continually removing any newly-formed orthotropic shoots or outgrowths from the pruned shoot, optionally with removing leaves and/or vegetative buds that form on the plagiotropic branch. The research underlying the invention has shown that the remaining plagiotropic branch develops at least one additional fork after a certain period of cultivating, which fork contains a new orthotropic shoot. By continually pruning these additional shoots, the time to flowering can be further accelerated, and/or the number of flowers and/or fruits generated per plagiotropic branch can be further increased. The continual removing can be carried out at least once every two months, preferably at least once a month, more preferably at least once every two weeks or at least once every week to ensure optimal conditions.

It is an advantage of the present invention that the time to flowering of the plant can be accelerated without hormone treatment and/or genetic manipulation. Thus, in a preferred embodiment, the method contains no hormone treatment and/or no genetic manipulation. A skilled person will of course understand that if desired the method of the invention can also include hormone treatment and/or genetically manipulated plants, but this is not required for the method of the invention to work.

The method of the invention can entail applying drought stress to the plant during cultivating, e.g., in step d. Drought stress can, e.g., be induced by reducing the applied volume of fertigation medium by at least 50%. The volume of fertigation medium can be reduced until the first symptoms of drought stress are observed on the plants, e.g., drooping, limp leaves, loss of turgidity etc. The drought stress can e.g., be applied to the plant for a period of 4-20 days. The inventors have found that as a part of the present invention, applying drought stress can further accelerate the time to flowering. Moreover, a timely application of drought stress can be used to synchronize the flowering between different plants.

Although not required, the method can comprise applying at least one plant hormone, which can be gibberellic acid, auxin, ethylene or any other phytohormone, to the plant during cultivating. The plant hormone can be applied to the plant continuously or in intervals ranging approximately from once a week to once a year. E.g., for gibberellic acid, dosages of from 0.1 to 200 mg per tree per application have proven useful. Preferably, the at least one plant hormone is applied by contacting a fluid containing the at least one plant hormone with the leaves of the plant, e.g., by spraying a hormone-containing solution onto the leaves of the plant.

The method according to the invention has the advantage that it allows for rapid flowering of the plant. The completion of steps b. to d. of the method of the invention, i.e. the time to flowering, can be 15 months or less, preferably 14 months or less, more preferably 13 months or less, 12 months or less, 11 months or less or even 10 months or less. Thus, the total time it takes from a rubiaceae plant seed or plant part to develop flowers capable of reproduction on the pruned shoot is considerably reduced as compared to the flowering time required in nature. For example, the time to obtain open flower (from b to d) is about 345 days for both treated and control plants.

A further advantage of the method of the invention is that it is particularly compatible with vertical gardening as it relies on outgrowth of only or mainly the first plagiotropic branch or branches. According to one embodiment, the cultivating of step b. and/or step d. is performed in a system in which the plants are arranged and cultivated in vertically stacked layers of culturing vessels, e.g., in flowerpots, and/or under soil-less growing conditions, e.g., using hydroponic techniques. Due to the pruning, only the at least one branch that will flower and produce fruits and seeds needs to be cultivated according to the method of the invention. This is particularly effective, as it takes up less space and allows for a vertical arrangement of the culturing vessels. In contrast, an unpruned plant of the same species takes up a much greater area, e.g., greater by 3.5-fold, and a greater volume, e.g., greater by 8-fold, than a plant which is pruned according to the invention, all while harboring less flowers per plagiotropic branch on average after 11-15 months of cultivating.

Surprisingly, the method of the invention not only leads to an accelerated and better controlled flowering, but it also leads to a higher number of flowers per cultivating time and plant mass. Furthermore, it was surprisingly found that the method of the invention allows for more than one flowering event per plant over the span of 1 year. At present, it is believed that the controlled lighting and/or continual pruning are responsible for this remarkable and very useful effect. Accordingly, the method of the invention can optionally comprise, subsequently to step d., it comprises a step e. of bud formation which occur 250 days after, preferably at 220 days after seed germination cutting for further rooting [[.]] of further cultivating the flowered shoot for a period of at maximum 8 months, until flowers form again on the flowered shoot. Furthermore, it is preferred that the method according to the invention optionally comprises subsequently to step d., it comprises a step e. of bud formation which occur 380 days after, preferably 340 days after seed sowing.

In particular the cultivating in step e. is performed until flowers form anew on the plagiotropic branch. Regarding the cultivating, all that has been described for steps b. and d. likewise applies to step e. With the method of the invention, the duration of step e, i.e. the time between the end of the first flowering to the second flowering on the plagiotropic branch, can be reduced to a period of at maximum 8 months, preferably at maximum 6 months, or at maximum 4 months. The further cultivating of step e. can be carried out with controlled lighting as e.g., in step d. and/or further continual pruning, with wholly or partially blocking off natural light. Good results were obtained with horticulture in a greenhouse at 16-35° C., 60-95% RH, and fertigation.

Due to the special pruning during cultivating and the very early flowering, the flowering plants obtained by the method of the invention are very different in many ways from plants of the family of rubiaceae known in the prior art to be in a flowering stage. Not only are the plants obtained by the method of the invention different in appearance due to the pruning and maintenance of only or mainly the plagiotropic branch or branches. They are also much younger at the time they flower as compared to flowering plants of the family of rubiaceae known in the prior art to be in a flowering stage.

Accordingly, the plant provided by the invention can be characterized in that it comprises at least one flower, preferably at least one flower per plagiotropic branch is at maximum 30 months of age, at maximum 29 months of age, at maximum 28 months of age, at maximum 27 months of age, at maximum 26 months of age or at maximum 25 months of age. The term “age” refers to the timespan between providing the plant seed or plant part which is capable of regeneration and the timepoint at which the first flower forms on the plant. Preferably the term “age” refers to the time between seed or seedling planting and the timepoint at which the first flower forms on the plant.

The pruning and/or controlled lighting according to the invention seems to be largely responsible for the acceleration of flowering in the plants. Accordingly, the pruning according to the invention can be used for accelerating the time of flowering of a plant of the family rubiaceae, wherein the pruning is carried out on a cultivated shoot of the plant at a growth stage in which it has branched into an orthotropic shoot and at least one plagiotropic branch, and wherein the pruning comprises removing at least the orthotropic shoot and maintaining at least one plagiotropic branch. With respect to the type of controlled lighting, the features described in connection with the cultivating of steps b. and d. of the method of the invention equally apply to the use of controlled lighting according to the invention to accelerate the time of flowering of a plant of the family rubiaceae, in particular of one of the genera Coffea.

### DESCRIPTION OF THE FIGURES

The invention will now be described with reference to the figure, which shall not be construed as limiting the scope or intent of the invention. The figure shows a schematical overview of the method according to the invention.

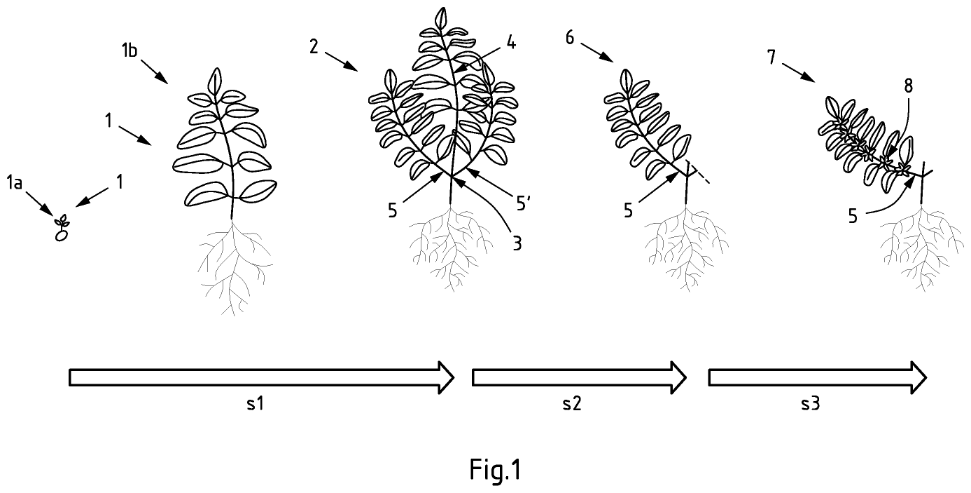

The FIG. 1 shows a schematical overview of the method according to the invention, which in a first step a. comprises providing a plant part 1 which is capable of regeneration. The plant part 1 can be a plant seed 1a or, e.g., a rooted plant cutting 1b. The plant part 1 is cultivated s1 in step b. of the method to obtain a cultivated shoot 2 which at a fork 3 branches into an orthotropic shoot 4 and two plagiotropic branches 5 and 5′. In step c. of the method, the cultivated shoot is pruned s2 by removing the orthotropic shoot 4 and all but one plagiotropic branch 5, i.e. in the case of the cultivated shoot 2 shown in the figure additionally the second plagiotropic branch 5′, thereby obtaining a pruned shoot 6 with at least one remaining plagiotropic branch 5. The cut point is shown for the pruned shoot 6 in the figure as a straight line. Subsequently to pruning s2, the pruned shoot 6 in step d. of the method of the invention is further cultivated s3 to obtain a flowered shoot 7 which according to the figure has one remaining plagiotropic branch 5 on which flowers 8 have formed.

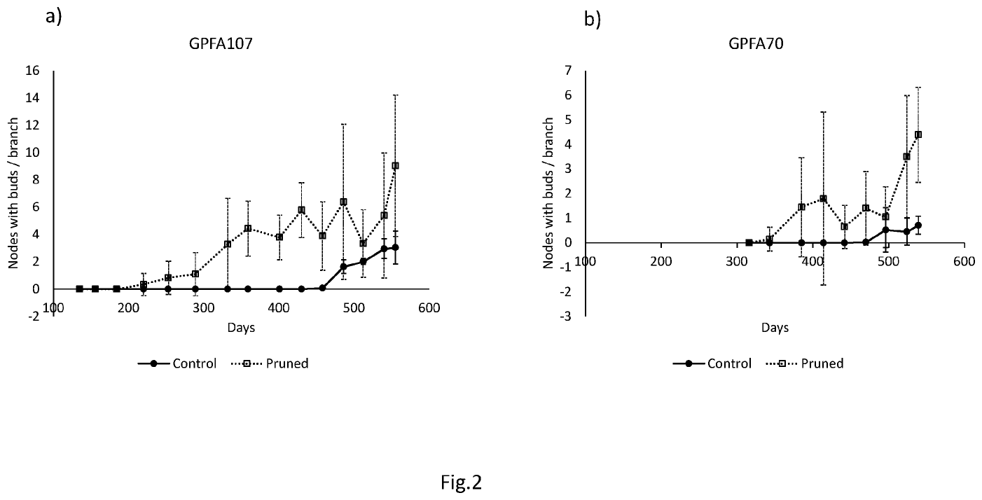

The FIG. 2 represents the average number of nodes where buds are observed per plagiotropic branch according to the type of plant management i.e. unpruned (control; continuous line) or pruned according to the method of the invention (dotted line) and according to the age of the plant expressed in days after either cutting for rooting (GPFA107; a) or seed sowing (GPFA70; b). Each dot represents a calculated mean of 3 or 20 measures for control and pruned plants, respectively. Vertical bars represent the standard errors.

### EXAMPLE: CULTIVATING A PLANT OF GENUS COFFEA

15 rooted cuttings of the genotype/variant GPFA107 of Coffea arabica were provided as plant parts capable of regeneration at the starting point of carrying out the method according to the invention. The date when the cutting were realized indicated the starting point for determining the plant age and was annoted t0. All rooted cuttings were cultivated in small soil-containing flowerpots in a greenhouse compartment indoors at 17-25° C., 72% RH. 10 of the rooted cuttings, also termed “group 1” were cultivated in vertical shelves, where lighting was provided by LED grow lamps (Philips Green Power Module Production 120 cm-Deep Red/White—50 μmol s⁻¹ each), which lights were arranged at a distance of at maximum 50 cm from the plants, with a photoperiod of 14 h per day. The remaining 5 rooted cuttings, also termed “group 2”, were cultivated on cultivating tables, with lighting by 4 high-pressure sodium vapor lamps, 400 W each, at a distance of approx. 2.5 m from the soil surface with a photoperiod of 12 h per day. During cultivating, the plant parts were fertigated by drippers twice daily.

Approximately once per month, the plants were checked for development of a fork, which would indicate that a cultivated shoot as defined herein had developed. Approx. 17-19 months after t0, a fork branching into an orthotropic shoot and at least one plagiotropic branch had appeared on each of the plants. The cultivated shoots of group 1 were pruned by removing the orthotropic shoot and all but one plagiotropic branch. The cuts were made at a distance of approx. 2-3 mm on the distal side of the fork. The cultivated shoots of group 2 were not pruned.

After pruning the plants of group 1, the plants of both groups were further cultivated until 30 months after t0. Starting at 25 months after t0, the number of floral buds, flowers and visible fruits was counted on each plant once a month. The first flowers appeared on the plants of group 1 after approx. 25-26 months of cultivation time.

Immediately after flower formation, all flowers were self-pollinated by hand. The number of pollinated flowers per plagiotropic branch was counted by determining whether seeds formed. Moreover, the number of visible fruits was counted on each plant. Numbers were averaged within each group, namely: group 1, which consists of pruned coffee plants; and group 2, which consists of unpruned coffee plants.

Table 1 shows the data for group 1 and group 2. “pp” means per plant; “pb” means per plagiotropic branch.

TABLE 1
Observations after cultivating.

| | | | no. of nodes | | |
|---|---|---|---|---|---|
| | | no. of | bearing | no. of | no. of |
| | | plagiotropic | floral | pollinated | visible |
| | cultivation | branches | buds | flowers | fruits |
| group | time | (pp) | (pb) | (pb) | (pb) |
| group 1 | 25 months | 1 | n.d. | 0.0 | 0.0 |
| pruned | 26 months | 1 | 3.3 | 25.6 | 0.0 |
| | 27 months | 1 | 1.4 | 25.6 | 3.4 |
| | 28 months | 1 | 1.9 | 26.1 | 18.5 |
| | 29 months | 1 | 3.2 | 26.1 | 20.7 |
| | 30 months | 1 | 3.8 | 26.1 | 17.9 |
| group 2 | 25 months | 20.2 | n.d. | 0.0 | 0.0 |
| not | 26 months | 22.8 | 0.3 | 0.2 | 0.0 |
| pruned | 27 months | 25.4 | 0.3 | 0.2 | 0.2 |
| | 28 months | 27.2 | 0.3 | 0.2 | 0.3 |
| | 29 months | 28.2 | 0.0 | 0.2 | 0.2 |
| | 30 months | 31.0 | 0.0 | 0.2 | 0.2 |

pp = per plant; pb = per plagiotropic branch.

The example shows that the pruning and/or controlled lighting according to the invention led to an increase in the number of total buds per plagiotropic branch. In particular, the number of nodes bearing floral buds showed a first maximum after 26 months, then decreased, then increased again, which indicates that after 2-3 months of cultivating the flowered shoot, a second generation of flowers started to form in the pruned plants. This effect was not observed in the unpruned plants.

Moreover, the pruning led to an increase in the number of nodes bearing floral buds per plagiotropic branch, and, more importantly, to an increase in the number of pollinated flowers and visible fruits per plagiotropic branch. Only by means of the method according to the invention, plants were generated that after 26 months of cultivating reliably harbored more than 1 flower per plagiotropic branch.

The example shows that by means of the method according to the invention, the number of fruits that can be obtained after a short period of cultivating can be significantly and reliably increased.

Finally, 296 seeds were isolated from the cherries collected on pruned trees and placed under germination conditions to assess viability. A total of 225 seeds germinated properly (76%) and were converted into plants indicating that the method is compatible with a use for coffee seed production.

### EXAMPLE

23 seedlings of GPFA70 (Arabica) as well as 23 cuttings of GPFA107 (also Arabica) were sown and grown until they reached the first plagiotropic ramification. For both varieties, 20 plants out of 23 were then pruned according to the method and placed inside growth chambers with controlled photoperiods (20 pruned trees, from 10 to 14 hours daylength, LED lighting 105 μmoles m⁻² s⁻¹ in average), whereas the remaining 3 plants were allowed to grow freely under greenhouse conditions (control: 3 unpruned trees under natural photoperiod with light compensation using HPS lamps delivering 50 μmoles·m⁻²·s⁻¹).

Plants were regularly checked for bud development at the node level and scored for the number of nodes showing bud development to measure the impact of the pruning combined with a LED lighting treatment. The density of buds per plagiotropic branch was also calculated to evaluate the impact of the pruning on this parameter.

The development of the first plagiotropic branches appeared 135 days after cuttings, whereas for GPFA70 seedlings, it was observable 273 days after sowing. It was found that there was an earlier bud induction for both varieties when pruned and grown under LED light compared to the control plants. Thus, buds appeared 153 and 197 days earlier on treated plants compared to control plants for GPFA70 and GPFA107, respectively (FIG. 2).

Earlier buds development was observed on GPFA107 pruned plants from rooted cutting compared to GPFA70 pruned plants from seedlings. This precocious development could not be observed on control plants.

Bud density per plagiotropic branch was found to be always higher for pruned plants compared to control, even if a higher variability could be observed.

1: A method for accelerating the time to flowering of and/or increasing a number of flowers formed by a plant of the family rubiaceae, the method comprising
a. providing a plant seed or plant part which is capable of regeneration,
b. cultivating the plant seed or plant part to a stage in which a cultivated shoot is obtained that comprises a fork from which an orthotropic shoot and at least two plagiotropic branches have developed,
c. pruning the cultivated shoot at said fork by removing at least the orthotropic shoot and maintaining at least one plagiotropic branch to obtain a pruned shoot,
d. cultivating the pruned shoot until flowers form on the at least one plagiotropic branch to obtain a flowered shoot.

2: The method according to claim 1, wherein the cultivated shoot obtained in step b. and pruned in step c. comprises only one fork, and/or the fork recited in step b. and used for pruning in step c. is the first fork that has developed in the cultivated shoot.

3: The method according to claim 1, wherein the cultivating in step d. further comprises continually removing any newly formed orthotropic shoots.

4: The method according to claim 1, further comprising subsequently to step d., a step e. of bud formation which occurs 250 days after cutting for further rooting.

5: The method according to claim 1, further comprising subsequently to step d., a step e. of bud formation which occurs 380 days after seed sowing.

6: The method according to claim 1, wherein the cultivating in step d. comprises stimulating specifically the at least one plagiotropic branch by exposure to a light source shining on the at least one plagiotropic branch, wherein the light source is positioned at a distance of 2 to 50 cm from a surface of the at least one plagiotropic branch and emits light in the spectral range of 400 to 750 nm.

7: The method according to claim 1, wherein the cultivating in step b. is carried out indoors and comprises lighting with a photoperiod of 8-14 h per day.

8: The method according to claim 1, wherein during the cultivating in step b. and/or step d., the lighting is set up such that the plant receives natural and/or artificial light having a photosynthetic photon flux density of 80 to 1500 μmol m⁻² s⁻¹.

9: The method according to claim 1, wherein the cultivating in step b. and/or step d. is carried out while wholly or partially blocking off natural light.

10: The method according to claim 9, wherein the blocking off of natural light is carried out only in step d.

11: The method according to claim 1, wherein the cultivating in step b. and/or step d. comprises growing at 16-35° C., 60-95% RH, and fertigation.

12: The method according to claim 1, wherein the method it contains no hormone treatment and no genetic manipulation.

13: The method according to claim 1, wherein the plant is of the genus Coffea.

14: A plant of the family rubiaceae comprising at least one flower per plagiotropic branch, obtained by the method according to claim 1, wherein the plant is at maximum 30 months of age.

15: The plant according to claim 14, wherein the plant is a variant exhibiting male sterility.

16: A method comprising using a plant of the family rubiaceae comprising at least one flower per plagiotropic branch for producing fruit and/or seeds or in a breeding program, wherein the plant is obtained by the method according to claim 1, wherein the plant is at maximum 30 months of age.

17: A method comprising using controlled lighting and/or pruning for accelerating time to flowering of and/or increasing the number of flowers formed by a plant of the family rubiaceae, wherein the pruning is carried out on a cultivated shoot of the plant at a growth stage in which the cultivated shoot has branched into an orthotropic shoot and at least two plagiotropic branches, and wherein the pruning comprises removing at least the orthotropic shoot and maintaining at least one plagiotropic branch.

18: The method according to claim 1, further comprising subsequently to step d., a step e. of bud formation which occurs 220 days after cutting for further rooting.

19: The method according to claim 1, further comprising subsequently to step d., a step e. of bud formation which occurs 340 days after seed sowing.

20: The method according to claim 7, wherein the cultivating in step b. and/or step d. is carried out while wholly or partially blocking off natural light, and the blocking off of natural light is carried out only in step d., only while outside the photoperiod.
